(19) United States
(12) Patent Application Publication
(10) Pub. No.: US 2011/0217381 A1
(43) Pub. Date: Sep. 8, 2011

(54) PHARMACEUTICAL COMPOSITIONS

(76) Inventors: Doris Angus, Liverpool (GB); David John Duncalf, Liverpool (GB); Alison Jane Foster, Liverpool (GB); Steven Paul Rannard, Liverpool (GB); Dong Wang, Liverpool (GB)

(21) Appl. No.: 13/061,533

(22) PCT Filed: Aug. 3, 2009
(86) PCT No.: PCT/EP09/60007

§ 371 Date: May 24, 2011

(30) Foreign Application Priority Data
Sep. 1, 2008 (GB) 0815852.9

(51) Int. Cl. A61K 9/14; A61K 31/4545; A61P 43/00

(52) U.S. Cl. 424/489; 514/290

(57) ABSTRACT

The present invention provides a substantially solvent-free nano-dispersion of an active in a carrier, wherein the carrier comprises an enteric carrier soluble at intestinal pH, but insoluble at stomach pH, and wherein the enteric carrier comprises at least 50% by weight of the nano-dispersion; and processes for the preparation of a substantially solvent-free nano-dispersion of an active in a carrier.

### FIELD OF THE INVENTION

The present invention relates to improvements relating to enteric-protected pharmaceutical compositions.

### BACKGROUND OF THE INVENTION

Enteric coatings are widely used for a variety of purposes including protection of acid sensitive pharmaceutical actives from stomach acid or protection of stomach mucous membranes from pharmaceuticals which cause irritation and or damage to the stomach wall.

For the enteric coating agent, the cellulose types including cellulose acetate phthalate (CAP), cellulose acetate trimellitate (CAT), hydroxypropylmethyl cellulose phthalate (HPMCP), hydroxypropylmethyl cellulose acetate succinate (HPMCAS) and carboxymethylethyl cellulose (CMEC), the vinyl types including polyvinyl alcohol acetate phthalate (PVAP) and the acrylic types including copolymers of methacrylic acid and ethyl acrylate are used.

Taking as an example the material HPMCP, the threshold pH value for rapid disintegration of HPMCP can be controlled by varying the phthalyl-content. It can therefore be ensured that while a tablet coated with HPMCP is insoluble in the stomach (where the pH is generally well below 4), it becomes soluble as it passes into the intestine (where the pH is typically well above 4). Thus, if a dosage unit of a pharmaceutical active, such as a tablet or capsule, is coated with HPMCP, the tablet or capsule will not release the active in the stomach, but will lose the coating and release the active in the intestine.

Our co-pending international patent application PCT/GB03/03226 (published as WO-2004/011537) describes the formation of solid, porous beads comprising a three dimensional open-cell lattice of a water-soluble polymeric material. These are typically ‘templated’ materials formed by the removal of both water and a non-aqueous dispersed phase from a high internal phase emulsion (HIPE) which has a polymer dissolved in the aqueous phase. The beads are formed by dropping the HIPE emulsion into a low temperature fluid such as liquid nitrogen, then freeze-drying the particles formed to remove the bulk of the aqueous phase and the dispersed phase. This leaves behind the polymer in the form of a ‘skeletal’ structure. The beads dissolve rapidly in water and have the property that a water-insoluble component dissolved in the non-aqueous phase of the emulsion prior to freezing and drying can also be dispersed in water on solution of the polymer skeleton of the beads.

WO-2006/079409 and WO-2008/006712 (which claim priority from GB 0501835.3 and GB 0613925.7, each filed 13 Jul. 2006) describe how materials which will form a nano-dispersion in water can be prepared, preferably by a spray-drying process. In WO-2006/079409, the water-insoluble materials are dissolved in the solvent-phase of an emulsion. In WO-2008/006712, the water-insoluble materials are dissolved in a mixed solvent system and co-exist in the same phase as a water-soluble structuring agent. In both cases the liquid is dried above ambient temperature (above 20° C.), such as by spray-drying, to produce particles of the structuring agent, as a carrier, with the water-insoluble materials dispersed therein. When these particles are placed in water they dissolve, forming a nano-dispersion of the water-insoluble material with particles typically below 300 nm. This scale is similar to that of virus particles, and the water-insoluble material behaves as though it were in solution.

WO-2008/007150 discloses both emulsion-based and single-phase methods of producing compositions comprising a water-insoluble paracetamol or non-steroidal anti-inflammatory drug (NSAID). The process comprises providing a mixture comprising the water-insoluble paracetamol or NSAID, a water-soluble carrier and a solvent for each of the paracetamol or NSAID and the carrier, and spray-drying the mixture to remove the or each solvent and obtain a substantially solvent-free nano-dispersion of the paracetamol or NSAID in the carrier. WO-2008/007150 does not disclose the use of a carrier that is soluble at intestinal pH, but insoluble at stomach pH.

WO-2005/020994 discloses a solid dispersion, especially a solid solution, comprising an active ingredient selected from tacrolimus and analogues thereof dispersed or dissolved in a hydrophilic or water-miscible vehicle, wherein the melting point of the vehicle is at least 20° C. and the active ingredient is present in a concentration of between about 0.01 w/w % and up to about 15 w/w %. WO-2005/020994 does not disclose a substantially solvent-free dispersion or a dispersion comprising an active and a carrier that is soluble at intestinal pH, but insoluble at stomach pH, or methods for preparing such dispersions.

EP-1741424 discloses a composition comprising a spray-dried solid dispersion comprising a sparingly-soluble drug and hydroxypropylmethylcellulose acetate succinate (HPMCAS), as well as a process for preparing the composition. There is no teaching in EP-1741424 to provide a nano-dispersion and the processes described therein do not produce nano-dispersions.

US-2007/0218138 discloses a solid dispersion comprising amorphous VX-950 (a competitive, reversible peptidomimetic HCV NS3/4A protease inhibitor) and a plurality of polymers. There is no disclosure in US-2007/0218138 of a nano-dispersion wherein a carrier that is soluble at intestinal pH, but insoluble at stomach pH comprises at least 50% by weight of the dispersion.

In the present application the term ‘ambient temperature’ means 20° C. and all percentages are percentages by weight unless otherwise specified.

### BRIEF DESCRIPTION OF THE INVENTION

We have now determined that both the emulsion-based and the single-phase method can be used to produce an enteric-protected water-soluble, disperse form of an active (such as a pharmaceutical active), by using a polymer which is insoluble in the stomach as the structuring agent. We have also found that known enteric-coating polymers are suitable for this purpose.

Accordingly, the present invention provides a substantially solvent-free nano-dispersion of an active in a carrier, wherein the carrier comprises an enteric carrier soluble at intestinal pH, but insoluble at stomach pH, and wherein the enteric carrier comprises at least 50% by weight of the nano-dispersion.

There is also provided a substantially solvent-free dispersion of a pharmaceutical or nutraceutical active in a carrier soluble at intestinal pH, but insoluble at stomach pH. Preferably, the dispersion is a nano-dispersion. Preferably, the active has solubility in water less than 10 g/L.

Accordingly, the present invention also provides a process for the production of a composition comprising an active, which process comprises the steps of:
(a) providing a mixture comprising:
   (i) said active,
   (ii) an enteric carrier soluble at intestinal pH, but insoluble at stomach pH, and
   (iii) a solvent for each of the active and the enteric carrier, and
(b) drying (preferably spray-drying) the mixture to remove the, or each, solvent and obtain a substantially solvent-free nano-dispersion of said active in said enteric carrier, wherein the enteric carrier comprises at least 50% by weight of the nano-dispersion.

There is also provided a process for the production of a composition comprising an active which comprises the steps of:
(a) providing a mixture comprising:
   (i) said active,
   (ii) a carrier soluble at intestinal pH, but insoluble at stomach pH,
   (iii) a solvent for each of the active and the carrier, and
(b) spray-drying the mixture to remove the, or each, solvent and obtain a substantially solvent-free dispersion of said active in said carrier. Preferably the dispersion of the active in the carrier is a nano-dispersion. Preferably, the active has a solubility in water is less than 10 g/L.

In embodiments of the invention, the active is either one which would be sensitive to the acidic conditions in the stomach, or one to which the stomach would itself be sensitive.

The dispersion of the active in the carrier is a nano-dispersion. A nano-dispersion, as the term is used herein, is a dispersion in which the average particle size is less than one micron. Preferably, the peak diameter of the active is below 800 nm. More preferably the peak diameter of the active is below 500 nm (for example between 350 and 450 nm). In a particularly preferred embodiment of the invention the peak diameter of the active is below 200 nm, most preferably below 100 nm.

The preferred method of particle sizing for the dispersed products of the present invention employs a dynamic light scattering instrument (Nano S, manufactured by Malvern Instruments UK). Specifically, the Malvern Instruments Nano S uses a red (633 nm) 4 mW Helium-Neon laser to illuminate a standard optical quality UV cuvette containing a suspension of material. The particle sizes quoted in this application are the “Z-average” diameter results obtained with that apparatus using the standard protocol. Particle sizes in solid products are the particle sizes inferred from the measurement of the particle size obtained by solution of the solid in water and measurement of the particle size.

It is believed that reduction of the particle size in the eventual nano-dispersion has significant advantages in improving the availability of the otherwise active material. This is believed to be particularly advantageous where an improved bio-availability is sought, or, in similar applications where high local concentrations of the material are to be avoided. Moreover it is believed that nano-dispersions with a small particle size are more stable than those with a larger particle size.

Any suitable active may be included in the nano-dispersion of the present invention. The active may be a pharmaceutical or a nutraceutical active. An active is an agent that is effective against a disorder or condition, such that when it is administered to a subject suffering from the disorder or condition it prevents and/or causes reduction, remission, or regression of the disorder or condition.

The active may be one that has a high solubility in aqueous solvents, such as water. This active is referred to throughout as a water-soluble active. In the context of the present invention, “high solubility” as applied to the active means that its solubility in water at neutral pH and at ambient temperature (20° C.) is greater than 10 g/L, preferably greater than 15 g/L. This solubility level provides the intended interpretation of what is meant by water-soluble in the present specification.

Alternatively, the active may be one that has a low solubility in aqueous solvents, such as water. In the context of the present invention, “low solubility” as applied to the active means that its solubility in water at neutral pH and at ambient temperature (20° C.) is less than 10 g/L. This active is referred to throughout as a water-insoluble active. Preferably, the water-insoluble active has solubility in water at neutral pH and at ambient temperature of less than 5 g/L preferably of less than 1 g/L, especially preferably of less than 150 mg/L, even more preferably of less than 100 mg/L. This solubility level provides the intended interpretation of what is meant by water-insoluble in the present specification.

Preferred actives include those falling in the class of pharmaceutical actives or food and nutraceuticals.

Examples of suitable water-insoluble actives and derivatives thereof include pharmaceutical actives such as antihypertensives (for example sartans, calcium channel blockers), NSAIDS, analgesics, lipid-regulating drugs (for example statins), anti-arrhythmic drugs (for example amiodarone), oral anti-diabetic drugs, anti-cancer drugs, antihistamines (for example loratadine, cetirizine), antipsycotics (for example olanzapine, haloperidol), antidepressants (for example amitriptyline, fluoxetine), anti-bacterials, anti-fungals, anti-virals, anti-parasitics, hormones, and nutraceuticals such as vitamins and vitamin-like substances (for example co-enzyme Q10).

Examples of suitable water-soluble actives and derivatives thereof include pharmaceutical actives such as water-soluble salts of the above listed water-insoluble actives and nutraceuticals such as water-soluble vitamins (for example vitamin C and vitamin B12).

The nano-dispersion comprises a carrier, which carrier comprises an enteric carrier soluble at intestinal pH, but insoluble at stomach pH.

The enteric carrier comprises at least 50% by weight, preferably at least 60% by weight, more preferably at least 70% by weight, even more preferably at least 80% by weight, of the nano-dispersion of the present invention (and of the nano-dispersions formed by the processes of the present invention). As the skilled person would appreciate enteric coating agents are typically used in low levels as coatings for pharmaceutical compositions. The present inventors have surprisingly appreciated that higher levels, i.e. at least 50% by weight, of such agents may instead be directly incorporated into dispersions as enteric carriers or structuring agents to achieve delivery of an active in the intestine and not in the stomach.

By an enteric carrier we mean a carrier or structuring agent that can effect enteric pH dissolution along the digestive tract. Any suitable enteric carrier may be used in the present invention, provided that it is soluble at intestinal pH, but insoluble at stomach pH. Preferred enteric carriers may be selected from the group consisting of enteric coating agents. Typically, these will be of the cellulose type (including cellulose acetate phthalate (CAP), cellulose acetate trimellitate (CAT), hydroxypropylmethyl cellulose phthalate (HPMCP, also known as hypromellose phthalate), hydroxypropylmethyl cellulose acetate succinate (HPMCAS) and carboxymethylethyl cellulose (CMEC)), the vinyl type (including polyvinyl alcohol acetate phthalate (PVAP)) and/or the acrylic type (including copolymers of methacrylic acid and ethyl acrylate).

The enteric carrier may include at least one of cellulose acetate phthalate, cellulose acetate trimellitate, hydroxypropylmethyl cellulose phthalate, hydroxypropylmethyl cellulose acetate succinate, carboxymethylethyl cellulose, polyvinyl alcohol acetate phthalate and/or copolymers of methacrylic acid and ethyl acrylate.

The enteric carrier may be selected from the group consisting of cellulose acetate phthalate, cellulose acetate trimellitate, hydroxypropylmethyl cellulose phthalate, hydroxypropylmethyl cellulose acetate succinate, carboxymethylethyl cellulose, polyvinyl alcohol acetate phthalate and copolymers of methacrylic acid and ethyl acrylate, and mixtures thereof.

Preferably, the enteric carrier may be of the cellulose type (including cellulose acetate phthalate, cellulose acetate trimellitate, hydroxypropylmethyl cellulose phthalate, hydroxypropylmethyl cellulose acetate succinate and carboxymethylethyl cellulose; especially cellulose acetate phthalate, cellulose acetate trimellitate, hydroxypropylmethyl cellulose phthalate, and carboxymethylethyl cellulose; more especially hydroxypropylmethyl cellulose phthalate).

In embodiments of the invention, the carrier may further comprise a water-soluble carrier and/or optionally one or more additional carriers, i.e. wherein the water-soluble carrier and/or optional additional carrier(s) are included in addition to the enteric carrier discussed herein. In particular, the carrier may further comprise a water-soluble carrier and optionally one or more additional carriers, i.e. wherein the water-soluble carrier and optional additional carrier(s) are included in addition to the enteric carrier discussed herein.

References herein to “carrier(s)” and “carrier material(s)” are intended to relate to all carriers included in the nano-dispersions, i.e. the enteric carrier and, when present, the water-soluble and additional carrier(s). References herein specifically to enteric carriers, water-soluble carriers and optional additional carriers are intended to relate to those specific carriers only.

In the context of the present invention, “high solubility” as applied to the carrier means that its solubility in water at neutral pH and at ambient temperature (20° C.) is greater than 10 g/L, preferably greater than 15 g/L. This solubility level provides the intended interpretation of what is meant by water-soluble in the present specification.

In the context of the present invention, “low solubility” as applied to the carrier means that its solubility in water at neutral pH and at ambient temperature (20° C.) is less than 10 g/L, preferably of less than 5 g/L, more preferably of less than 1 g/L, especially preferably of less than 150 mg/L, even more preferably of less than 100 mg/L. This solubility level provides the intended interpretation of what is meant by water-insoluble in the present specification.

As the skilled person would appreciate, when a water-soluble carrier, and optionally one or more additional carriers, are present in the processes of the present invention these carriers may be combined in the mixture so formed, i.e. such that the mixture further comprises a water-soluble carrier and optionally one or more additional carriers. In this aspect, the mixture may therefore comprise the active, enteric carrier, water-soluble carrier, optional additional carrier(s) and a solvent for the active and for each of the enteric, water-soluble and optional additional carriers.

The process of the present invention may comprise forming a mixture in the form of an emulsion. Such a process may be used to prepare a nano-dispersion comprising a water-soluble active. A further aspect of the present invention therefore provides a process for the production of a composition comprising a water-soluble active, which process comprises the steps of:
(a) providing a mixture by forming an emulsion comprising:
   (i) a solution of the water-soluble active in water, and
   (ii) a solution of an enteric carrier in a water immiscible solvent for the same, and,
(b) drying (preferably spray-drying) the emulsion to remove water and the water-immiscible solvent to obtain the substantially solvent-free nano-dispersion of the active in the enteric carrier, wherein the enteric carrier comprises at least 50% by weight of the nano-dispersion.

There is also provided a process for the production of a composition comprising an active which comprises the steps of:
(a) forming an emulsion comprising:
   (i) a solution of the active in water, and
   (ii) a solution of the carrier in a water immiscible solvent for the same, and,
(b) drying the emulsion to remove water and the water-immiscible solvent to obtain a substantially solvent-free dispersion of the active in the carrier.

For convenience, this class of method is referred to herein as the “emulsion” method. Optionally, the emulsion may further comprise a water-soluble carrier and/or optionally one or more additional carriers as discussed herein. Optionally, the emulsion may further comprise a water-soluble carrier and optionally one or more additional carriers as discussed herein, for example wherein the water-soluble carrier is comprised in the aqueous solution (i) and the optional additional carrier(s) may be comprised in the aqueous solution (i) and/or the water-immiscible solvent solution (ii) according to their type. Thus, the emulsion may further comprise a water-soluble carrier in the aqueous solution (i) and optionally one or more additional carriers (i.e. in the solution (i) and/or (ii) as appropriate).

The process of the present invention may comprise forming a mixture in the form of a single phase mixture. Such a process is compatible with both water-soluble and water-insoluble actives. A further aspect of the present invention therefore provides a process for the production of a composition comprising an active (said active being water-insoluble or water-soluble), which process comprises the steps of:
(a) providing a mixture by forming a single phase mixture comprising:
   (i) at least one non-aqueous solvent
   (ii) optionally, water
   (iii) an enteric carrier soluble in the mixture of (i) and (ii) and
   (iv) said active which is soluble in the mixture of (i) and (ii), and,
(b) drying (preferably spray-drying) the solution to remove any water and the water miscible solvent to obtain the substantially solvent-free nano-dispersion of the active in the enteric carrier, wherein the enteric carrier comprises at least 50% by weight of the nano-dispersion.

There is also provided a process for the production of a composition comprising an active which comprises the steps of:
(a) providing a single phase mixture comprising:
   (i) at least one non-aqueous solvent
   (ii) optionally, water
   (iii) said carrier material soluble in the mixture of (i) and (ii) and
   (iv) said active which is soluble in the mixture of (i) and (ii), and,
(b) drying the solution to remove any water and the water miscible solvent to obtain a substantially solvent-free dispersion of the active in the carrier.

For convenience, this class of method is referred to herein as the “single-phase” method. Optionally, the single phase mixture may further comprise a water-soluble carrier and/or optionally one or more additional carriers as discussed herein. Optionally, the single phase mixture may further comprise a water-soluble carrier and optionally one or more additional carriers as discussed herein, for example wherein the water-soluble carrier and the optional additional carrier(s) are soluble in the mixture of (i) and (ii). Thus, the single phase mixture may further comprise a water-soluble carrier and optionally one or more additional carriers soluble in the mixture of (i) and (ii).

In the context of the present invention “substantially solvent-free” means that the free solvent content of the product is less than 15% wt, preferably below 10% wt, more preferably below 5% wt and most preferably below 2% wt.

In the context of the present invention, it is essential that both the carrier(s) and the active are essentially fully dissolved in their respective solvents prior to the drying step. It is not within the ambit of the present specification to teach the drying of slurries. For the avoidance of any doubt, it is therefore the case that the solids content of the emulsion or the mixture is such that over 90% wt, preferably over 95% wt, and more preferably over 98% wt, of the soluble materials present is in solution prior to the drying step.

In relation to the processes mentioned above, the preferred active and the preferred carrier(s) are as described above and as elaborated on in further detail below. Similarly the preferred physical characteristics of the material are as described above.

The ‘single phase’ method where both the active and the carrier(s) are dissolved in a phase comprising at least one non-aqueous solvent (and optional water) is preferred. This is believed to be more efficacious in obtaining a smaller particle size for the nano-disperse active. Preferably, the drying step simultaneously removes both the water and other solvents and, more preferably, drying is accomplished by spray drying at above ambient temperature.

A further aspect of the present invention provides a pharmaceutical product or composition comprising a substantially solvent-free nano-dispersion as described herein.

A further aspect of the present invention provides a nutraceutical product or composition comprising a substantially solvent-free nano-dispersion as described herein.

A further aspect of the present invention provides the use of a substantially solvent-free nano-dispersion as described herein for the delayed release of the active to a subject upon administration of the nano-dispersion to the subject.

A further aspect of the present invention provides the use of a substantially solvent-free nano-dispersion as described herein as a delayed release medicament or nutraceutical.

A further aspect of the present invention provides a substantially solvent-free nano-dispersion as described herein for use in the delayed release of the active upon administration to a subject, i.e. to treat and/or prevent a disease or condition in the subject.

A further aspect of the present invention provides the use of a substantially solvent-free nano-dispersion as described herein in the manufacture of a medicament for use in treating and/or preventing a disorder or condition in a subject, i.e. through delayed release of the active to the subject.

A further aspect of the present invention provides the use of a substantially solvent-free nano-dispersion as described herein in the manufacture of a nutraceutical for use in treating and/or preventing a disorder or condition in a subject, i.e. through delayed release of the active to the subject.

A further aspect of the present invention provides a method for treating and/or preventing a disorder or condition in a subject in need thereof (i.e. through delayed release of the active to the subject), which method comprises administering to said subject a therapeutically effective amount of a substantially solvent-free nano-dispersion as described herein.

A further aspect of the present invention provides the use of a product or composition as described herein for the delayed release of the active to a subject upon administration of the product or composition to the subject.

A further aspect of the present invention provides the use of a product or composition as described herein as a delayed release medicament or nutraceutical.

A further aspect of the present invention provides a product or composition as described herein for use in the delayed release of the active upon administration to a subject, i.e. to treat and/or prevent a disease or condition in the subject.

A further aspect of the present invention provides the use of a product or composition as described herein in the manufacture of a medicament for use in treating and/or preventing a disorder or condition in a subject, i.e. through delayed release of the active to the subject.

A further aspect of the present invention provides the use of a product or composition as described herein in the manufacture of a nutraceutical for use in treating and/or preventing a disorder or condition in a subject, i.e. through delayed release of the active to the subject.

A further aspect of the present invention provides a method for treating and/or preventing a disorder or condition in a subject in need thereof (i.e. through delayed release of the active to the subject), which method comprises administering to said subject a therapeutically effective amount of a product or composition as described herein.

By “delayed release” we mean that the active is released in the intestine, with no substantial release in the stomach.

A further aspect of the present invention provides a kit comprising a substantially solvent-free nano-dispersion as described herein.

A further aspect of the present invention provides a kit comprising a product or composition as described herein.

A further aspect of the present invention provides a method for the preparation of a medicament for use in the treatment of disease which comprises the step of preparing a composition according to the present invention.

As will be noted hereafter, materials prepared according to the present invention show slow release under acid conditions and rapid release in alkaline solutions.

### DETAILED DESCRIPTION OF THE INVENTION

Various preferred features and embodiments of the present invention are described in further detail below.

### Actives:

As discussed above, preferred actives include those falling in the class of pharmaceutical actives or food and nutraceuticals. Examples of suitable water-insoluble actives include pharmaceutical actives such as antihypertensives (for example sartans, calcium channel blockers), NSAIDS, analgesics, lipid-regulating drugs (for example statins), anti-arrhythmic drugs (for example amiodarone), oral anti-diabetic drugs, anti-cancer drugs, antihistamines (for example loratadine, cetirizine), antipsycotics (for example olanzapine, haloperidol), antidepressants (for example amitriptyline, fluoxetine), anti-bacterials, anti-fungals, anti-virals, anti-parasitics and hormones, and nutraceuticals such as vitamins and vitamin-like substances (for example co-enzyme Q10).

Examples of suitable water-soluble actives and derivatives thereof include pharmaceutical actives such as water-soluble salts of the above listed water-insoluble actives and nutraceuticals such as water-soluble vitamins (for example vitamin C and vitamin B12).

### Product Form:

The structure of the material obtained after the drying step is not well understood. It is believed that the resulting dry materials are not encapsulates, as discrete macroscopic bodies of the actives are not present in the dry product. It is also believed that the compositions are not so-called solid solutions, as with the present invention the ratios of components present can be varied without loss of the benefits. Also from X-ray and DSC studies, it is believed that the compositions of the invention are not solid solutions, but comprise nano-scale, phase-separated mixtures.

Preferably, the compositions produced after the drying step will comprise the active and the carrier in a weight ratio of from 1:500 to 4:5 (as active:carrier), 1:100 to 4:5 being preferred. Typical levels of around 10 to 50% wt, particularly 10 to 30% wt, active and 90 to 50% wt, particularly 90 to 70% wt, carrier can be obtained by spray-drying.

In particular, the substantially solvent-free nano-dispersions of the present invention may comprise from 10 to 50% wt of the active and from 50 to 90% wt of the carrier, wherein the carrier comprises an enteric carrier (i.e. which is soluble at intestinal pH, but insoluble at stomach pH) and wherein the enteric carrier comprises at least 50% by weight of the nano-dispersion.

In particular, the substantially solvent-free nano-dispersions of the present invention may comprise from 10 to 35% wt of the active and from 65 to 90% wt of the carrier, wherein the carrier comprises an enteric carrier (i.e. which is soluble at intestinal pH, but insoluble at stomach pH) and wherein the enteric carrier comprises at least 50% by weight of the nano-dispersion.

More particularly, the substantially solvent-free nano-dispersions of the present invention may comprise from 10 to 30% wt of the active and from 70 to 90% wt of the carrier, wherein the carrier comprises an enteric carrier (i.e. which is soluble at intestinal pH, but insoluble at stomach pH) and wherein the enteric carrier comprises at least 50% by weight of the nano-dispersion.

When the carrier comprises a water-soluble carrier in addition to the enteric carrier, the substantially solvent-free nano-dispersions of the present invention may particularly comprise from 10 to 30% wt of the active and from 70 to 90% wt of the carrier, wherein the carrier comprises an enteric carrier (i.e. which is soluble at intestinal pH, but insoluble at stomach pH) and a water-soluble carrier and wherein the enteric carrier comprises from 50% wt to 80% wt by weight of the nano-dispersion and the remainder of the carrier comprises the water-soluble carrier.

When the carrier comprises a water-soluble carrier and optionally one or more additional carriers in addition to the enteric carrier, the substantially solvent-free nano-dispersions of the present invention may particularly comprise from 10 to 30% wt of the active and from 70 to 90% wt of the carrier, wherein the carrier comprises an enteric carrier (i.e. which is soluble at intestinal pH, but insoluble at stomach pH), a water-soluble carrier and optionally one or more additional carriers and wherein the enteric carrier comprises from 50% wt to 80% wt by weight of the nano-dispersion and the remainder of the carrier comprises the water-soluble carrier and optionally the one or more additional carriers.

By the method of the present invention the particle size of the actives can be reduced to below 500 nm and may be reduced to around 300 nm. Preferred particle sizes are in the range of 40 to 300 nm.

### ‘Emulsion’ Preparation Method:

In one preferred process according to the invention the solvent for the enteric carrier is not miscible with water. On admixture with water (containing the active) it therefore can form an emulsion.

Preferably, the discontinuous (e.g. aqueous) phase comprises from about 10% to about 95% v/v of the emulsion, more preferably from about 20% to about 68% v/v.

The emulsions are typically prepared under conditions which are well known to those skilled in the art, for example, by using a magnetic stirring bar, a homogeniser, or a rotational mechanical stirrer. The emulsions need not be particularly stable, provided that they do not undergo extensive phase separation prior to drying.

Homogenisation using a high-shear mixing device (e.g. a jet homogeniser) is a particularly preferred way to make an emulsion in which the aqueous phase is the discontinuous phase. It is believed that this avoidance of coarse emulsion and reduction of the droplet size of the dispersed phase of the emulsion, results in an improved dispersion of the active material in the dry product.

In a preferred method according to the invention an emulsion is prepared with an average dispersed (aqueous)—phase droplet size (using the Malvern Z-average peak intensity) of between 500 nm and 5000 nm. We have found that an ‘Ultra-Turrux’ T25 type laboratory homogenizer (or equivalent) gives a suitable emulsion when operated for more than a minute at above 10,000 rpm.

There is a directional relation between the emulsion droplet size and the size of the particles of the active, which can be detected after dispersion of the materials of the invention in phosphate buffered saline solution. We have determined that an increase in the speed of homogenization for precursor emulsions can decrease final particle size after re-dissolution.

It is believed that the re-dissolved particle size can be reduced by nearly one half when the homogenization speed increased from 13,500 rpm to 21,500 rpm. The homogenization time is also believed to play a role in controlling re-dissolved particle size. The particle size again decreases with increase in the homogenization time, and the particle size distribution become broader at the same time.

Sonication is also a particularly preferred way of reducing the droplet size for emulsion systems. We have found that a Heat Systems Sonicator XL operated at level 10 for two minutes is suitable or a Hielscher UP400S operated with an amplitude of 60 or 80 for 3 minutes.

It is believed that ratios of components which decrease the relative concentration of the active and/or the carrier give a smaller particle size.

### ‘Single Phase’ Preparation Method:

In an alternative method according to the present invention both the carrier(s) and the active are soluble in a non-aqueous solvent or a mixture of such a solvent with water. Both here and elsewhere in the specification the non-aqueous solvent can be a mixture of non-aqueous solvents.

In this case the feedstock of the drying step can be a single phase material in which both the carrier and the active are dissolved. It is also possible for this feedstock to be present as one component of an emulsion, provided that both the carrier and the active are dissolved in the same phase.

The ‘single-phase’ method is generally believed to give a better nano-dispersion with a smaller particle size than the emulsion method.

It is believed that ratios of components which decrease the relative concentration of the active to the solvents and/or the carrier give a smaller particle size.

### Drying:

Any suitable drying method may be used in the processes of the present invention. In particular, spray-drying may be used.

Spray-drying is well known to those versed in the art. In the case of the present invention some care must be taken due to the presence of a volatile non-aqueous solvent in the emulsion being dried. In order to reduce the risk of explosion when a flammable solvent is being used, an inert gas, for example nitrogen, can be employed as the drying medium in a so-called closed spray-drying system. The solvent can be recovered and re-used.

We have found that the ‘Buchr’ B-290 type laboratory spray drying apparatus is suitable.

It is preferable that the drying temperature should be at or above 60° C., preferably above 80° C., more preferably above 100° C. For example, suitable temperature ranges may be from 60° C. to 130° C., especially from 80° C. to 130° C. Elevated drying temperatures have been found to give smaller particles in the re-dissolved nano-disperse material.

Other drying methods well know to those versed in the art can be used. Typical methods include freeze-drying or spray granulation.

### Carrier:

The enteric carrier is soluble in the intestine, which includes the formation of structured aqueous phases as well as true ionic solution of molecularly mono-disperse species. As noted above suitable enteric carriers include enteric-protective materials of:
a) the cellulose type (including:
   cellulose acetate phthalate (CAP),
   cellulose acetate trimellitate (CAT),
   hydroxypropylmethyl cellulose phthalate (HPMCP),
   hydroxypropylmethyl cellulose acetate succinate (HPMCAS), and
   carboxymethylethyl cellulose (CMEC)), and/or,
b) the vinyl types (including polyvinyl alcohol acetate phthalate (PVAP)), and/or,
c) the acrylic types (including copolymers of methacrylic acid and ethyl acrylate), and/or
d) mixtures thereof.

As discussed above, it is envisaged that water-soluble carriers may be present in addition to the enteric carrier. However, the levels of these should be such that they do not interfere with the protective effect of the enteric carrier in the stomach. Typically of the total material present, some 50 to 90% wt, particularly some 65 to 90% wt, more particularly some 70 to 90% wt, will be the carrier, with at least 50% wt being enteric carrier.

Suitable water-soluble carriers include polymers (preferably polyols, such as polyvinylalcohol) other than enteric protecting agents and/or surfactants. It is preferred that these other polymers do not exceed 20% wt of the composition.

Where the other water-soluble carriers include surfactant, the surfactant may be non-ionic, anionic, cationic, amphoteric or zwitterionic.

Examples of suitable non-ionic surfactants include ethoxylated triglycerides; fatty alcohol ethoxylates; alkylphenol ethoxylates; fatty acid ethoxylates; fatty amide ethoxylates; fatty amine ethoxylates; sorbitan alkanoates; ethylated sorbitan alkanoates; alkyl ethoxylates; Pluronics™, alkyl polyglucosides; stearol ethoxylates; alkyl polyglycosides.

Examples of suitable anionic surfactants include alkylether sulfates; alkylether carboxylates; alkylbenzene sulfonates; alkylether phosphates; dialkyl sulfosuccinates; sarcosinates; alkyl sulfonates; soaps; alkyl sulfates; alkyl carboxylates; alkyl phosphates; paraffin sulfonates; secondary n-alkane sulfonates; alpha-olefin sulfonates; isethionate sulfonates.

Examples of suitable cationic surfactants include fatty amine salts; fatty diamine salts; quaternary ammonium compounds; phosphonium surfactants; sulfonium surfactants; sulfonxonium surfactants.

Examples of suitable zwitterionic surfactants include N-alkyl derivatives of amino acids (such as glycine, betaine, aminopropionic acid); imidazoline surfactants; amine oxides; amidobetaines.

Mixtures of water-soluble carriers may be used. Mixtures of surfactants may be used. In such mixtures there may be individual components which are liquid, provided that the carrier material overall, is a solid.

Alkoxylated nonionic's (especially the PEG/PPG Pluronic™ materials), phenol-ethoxylates (especially TRITON™ materials), alkyl sulfonates (especially SDS), ester surfactants (preferably sorbitan esters of the Span™ and Tween™ types) and cationics (especially cetyltrimethylammonium bromide—CTAB) are particularly preferred as adjuncts to the enteric carrier.

Typical levels of surfactant in compositions according to the invention will be 5 to 15% wt of dry matter. In one particularly preferred embodiment the composition comprises 5 to 15% wt of an alkoxylated nonionic surfactant, 65 to 85% wt of HPMCP and 5 to 15% wt of the active.

Examples of suitable water-soluble polymeric carrier materials include:
(a) natural polymers (for example naturally occurring gums such as guar gum, alginate, locust bean gum or a polysaccharide such as dextran);
(b) cellulose derivatives for example xanthan gum, xyloglucan, cellulose acetate, methylcellulose, methyl-ethylcellulose, hydroxy-ethylcellulose, hydroxy-ethylmethyl-cellulose, hydroxy-propylcellulose, hydroxy-propylmethylcellulose, hydroxy-propylbutylcellulose, ethylhydroxy-ethylcellulose, carboxy-methylcellulose and its salts (for example the sodium salt—SCMC), or carboxy-methylhydroxyethylcellulose and its salts (for example the sodium salt);
(c) homopolymers of or copolymers prepared from two or more monomers selected from: vinyl alcohol, acrylic acid, methacrylic acid, acrylamide, methacrylamide, acrylamide methylpropane sulphonates, aminoalkylacrylates, aminoalkyl-methacrylates, hydroxyethylacrylate, hydroxyethylmethylacrylate, vinyl pyrrolidone, vinyl imidazole, vinyl amines, vinyl pyridine, ethyleneglycol and other alkylene glycols, ethylene oxide and other alkylene oxides, ethyleneimine, styrenesulphonates, ethyleneglycolacrylates and ethyleneglycol methacrylate;
(e) cyclodextrins, for example beta-cyclodextrin; and
(f) mixtures thereof.

When the polymeric material is a copolymer it may be a statistical copolymer (heretofore also known as a random copolymer), a block copolymer, a graft copolymer or a hyperbranched copolymer. Co-monomers other than those listed above may also be included in addition to those listed if their presence does not destroy the water-soluble nature of the resulting polymeric material.

Examples of suitable and preferred homopolymers include poly-vinylalcohol, poly-acrylic acid, poly-methacrylic acid, poly-acrylamides (such as poly-N-isopropylacrylamide), poly-methacrylamide; poly-acrylamines, poly-methyl-acrylamines, (such as polydimethylaminoethylmethacrylate and poly-N-morpholinoethylmethacrylate), polyvinylpyrrolidone, poly-styrenesulphonate, polyvinylimidazole, polyvinylpyridine, poly-2-ethyl-oxazoline poly-ethyleneimine and ethoxylated derivatives thereof.

Polyethylene glycol (PEG), polyvinylpyrrolidone (PVP), poly(2-ethyl-2-oxazaline), polyvinyl alcohol (PVA) hydroxypropyl cellulose and hydroxypropyl-methyl cellulose (HPMC) and alginates are preferred water-soluble polymeric carrier materials.

Further examples of suitable water-soluble carriers include water-soluble inorganic materials which are neither a surfactant nor a polymer. Simple organic salts have been found suitable, particularly in admixture with polymeric and/or surfactant carriers as described above. Suitable salts include carbonate, bicarbonates, halides, sulfates, nitrates and acetates, particularly soluble salts of sodium, potassium and magnesium. Preferred materials include, sodium carbonate, sodium bicarbonate and sodium sulphate. These materials have the advantage that they are cheap and physiologically acceptable. They are also relatively inert as well as compatible with many materials found in pharmaceutical and nutraceutical products.

Mixtures of water-soluble carrier materials are advantageous. Preferred mixtures include combinations of surfactants and polymers for example that include at least one of:
(a) Polyethylene glycol (PEG), polyvinylpyrrolidone (PVP), hydroxypropyl cellulose and hydroxypropyl-methyl cellulose (HPMC), alginates and, at least one of;
b) Alkoxylated nonionic's (especially the PEG/PPG Pluronic™ materials), phenol-ethoxylates (especially TRITON™ materials), alkyl sulphonates (especially SDS), ester surfactants (preferably sorbitan esters of the Span™ and Tween™ types) and cationics (especially cetyltrimethylammonium bromide—CTAB).

Further examples of suitable water-soluble carriers include water-soluble small organic materials which are neither a surfactant, a polymer nor an inorganic carrier material. Simple organic sugars have been found to be suitable, particularly in admixture with a water-soluble polymeric and/or surfactant carrier material as described above. Suitable small organic materials include mannitol, polydextrose, xylitol and inulin etc.

Any suitable additional carrier(s) may optionally be included in the dispersions discussed herein. Mixtures of additional carriers may be used.

Examples of suitable additional carriers include water-insoluble non-enteric carriers such as alkyl methacrylates (for example poly(methyl methacrylate)), polyesters (for example poly(caprolactone)), poly(vinyl acetate), poly(styrene) and co-polymers of these, waxes, viscous oils (for example paraffin wax, carnauba wax, paraffin oil, siloxanes), poorly water-soluble alcohols, fatty acids and surfactants (for example cetyl alcohol, stearic acid and sorbitan esters).

Non-aqueous solvent:

In those embodiments of the invention in which the dryer feedstock comprises a volatile, second non-aqueous solvent, this may either be miscible with the other solvents in pre-mix before drying or, together with those solvents may form an emulsion.

In one alternative form of the invention a single, non-aqueous solvent is employed in which can form a single phase with water in the presence of the active and the carrier. Preferred solvents for these embodiments are polar, protic or aprotic solvents. Generally preferred solvents have a dipole moment greater than 1 and a dielectric constant greater than 4.5.

Particularly preferred solvents are selected from the group consisting of haloforms (preferably dichloromethane, chloroform), lower (C1-C10) alcohols (preferably methanol, ethanol, isopropanol, isobutanol), organic acids (preferably formic acid, acetic acid), amides (preferably formamide, N,N-dimethylformamide), nitriles (preferably aceto-nitrile), esters (preferably ethyl acetate) aldehydes and ketones (preferably methyl ethyl ketone, acetone), and other water miscible species comprising hetroatom bond with a suitably large dipole (preferably tetrahydrofuran, dialkylsulfoxide).

Haloforms, lower alcohols, ketones and dialkylsulfoxides are the most preferred solvents.

A mixture of non-aqueous solvents may be used, for example an ethanol/acetone mix.

In another alternative form of the invention the non-aqueous solvent is not miscible with water and forms an emulsion.

The non-aqueous phase of the emulsion is preferably selected from one or more from the following group of volatile organic solvents:
alkanes, preferably heptane, nhexane, isooctane, dodecane, decane;
cyclic hydrocarbons, preferably toluene, xylene, cyclohexane;
halogenated alkanes, preferably dichloromethane, dichoroethane, trichloromethane (chloroform), fluoro-trichloromethane and tetrachloroethane;
esters preferably ethyl acetate;
ketones preferably 2-butanone;
ethers preferably diethyl ether;
volatile cyclic silicones preferably either linear or cyclomethicones containing from 4 to 6 silicon units. Suitable examples include DC245 and DC345, both of which are available from Dow Corning Inc.

Preferred solvents include dichloromethane, chloroform, ethanol, acetone and dimethyl sulfoxide.

Preferred non-aqueous solvents, whether miscible or not, have a boiling point of less than 150° C. and, more preferably, have a boiling point of less than 100° C., so as to facilitate drying, particularly spray-drying under practical conditions and without use of specialised equipment. Preferably they are non-flammable, or have a flash point above the temperatures encountered in the method of the invention.

Preferably, the non-aqueous solvent comprises from about 10% to about 95% v/v of any emulsion formed, more preferably from about 20% to about 80% v/v. In the single phase method the level of solvent is preferably 20 to 100% v/v.

Particularly preferred solvents are alcohols, particularly ethanol and halogenated solvents, more preferably chlorine-containing solvents, most preferably solvents selected from (di- or tri-chloromethane).

### Optional Cosurfactant:

In addition to the non-aqueous solvent an optional co-surfactant may be employed in the composition prior to the drying step. Preferred co-surfactants are short chain alcohols or amine with a boiling point of <220° C.

Preferred co-surfactants are linear alcohols. Preferred co-surfactants are primary alcohols and amines. Particularly preferred co-surfactants are selected from the group consisting of the 3 to 6 carbon alcohols. Suitable alcohol co-surfactants include n-propanol, n-butanol, n-pentanol, n-hexanol, hexylamine and mixtures thereof.

Preferably the co-surfactant is present in a quantity (by volume) less than the solvent preferably the volume ratio between the solvent and the co-surfactant falls in the range 100:40 to 100:2, more preferably 100:30 to 100:5.

### Pharmaceutical/Nutraceutical Product or Composition

The dispersion of the present invention may be formulated as a pharmaceutical or nutraceutical product or composition, i.e. in a form suitable for administration to a subject.

The pharmaceutical or nutraceutical product or composition may be formulated for administration to a subject by any suitable means, especially for oral administration.

For example, the pharmaceutical products or compositions may be in a form suitable for oral administration such as in a solid dosage form, for example as tablets or capsules. Solid dosage forms can include one or more substances which may also act as flavoring agents, lubricants, solubilizers, suspending agents, fillers, glidants, compression aids, binders or tablet-disintegrating agents.

The “subject” to which the dispersion and/or pharmaceutical or nutraceutical product/composition of the invention may be administered is an animal, especially a warm-blooded animal, such as a domestic animal or man, particularly man.

### EXAMPLES

In order that the present invention may be further understood and carried forth into practice it is further described below with reference to non-limiting examples.

### Example 1

0.10 g Loratadine and 0.70 g hypromellose phthalate (HPMCP) were dissolved into 80 ml ethanol/acetone mixture (50% v/v); 0.10 g pluronic F127 and 0.10 g mannitol were dissolved into 20 ml distilled water. The water solution was then added into the ethanol/acetone mixture and stirred using a magnetic bar to form a homogeneous solution. The solution was spray dried using a Buchi Mini B-290 spray dryer with an inlet temperature of 150° C. and liquid feed rate at 2.5 ml/min. A free-flowing white powder was obtained.

A sample of the dried powder was re-dispersed into phosphate buffer solution (pH=7.2) and the nanoparticle size was measured using a Malvern Nano-S. A particle size measurement of 385±21 nm. (at 5 mg/ml concentration) was obtained (after correcting for viscosity).

### Example 2

0.10 g Loratadine and 0.80 g hypromellose phthalate (HPMCP) were dissolved into 80 ml ethanol/acetone mixture (50%, v/v); 0.10 g pluronic F127 was dissolved into 10 ml distilled water. The water solution was then added into the ethanol/acetone mixture and stirred using a magnetic bar to form a homogeneous solution. The mixture was spray dried using a Buchi Mini B-290 spray dryer with an inlet temperature of 150° C. and liquid feed rate at 2.5 ml/min. A free-flowing white powder was obtained.

A sample of the dried powder was re-dispersed into phosphate buffer solution (pH=7.2) and the nanoparticle size was measured using a Malvern Nano-S. A particle size measurement of 429±8 nm (at 5 mg/ml concentration) was obtained (without viscosity correction).

### Example 3

### Dissolution test for Example 1

100 mg (equivalent to 10 mg loratadine) of the spray dried powder from Example 1 was dispersed into 900 ml dissolution media (either distilled water, HCl solution at a pH=2.2, or phosphate buffer solution at a pH=7.2 respectively) with overhead paddle stirring at 50 rpm and a temperature of 37° C. (of the dissolution media). Aliquots of each solution were taken using a pipette (1 ml Eppendorf pipette) at 5 min, 10 min, 20 min, etc. The dispersions were then diluted with ethanol for UV characterization (1 ml ethanol was added into 1 ml dispersion). The dissolution of Example 1 in different media is summarized in Table 1.

TABLE 1

| | | % release in acid | % release in buffer, |
|---|---|---|---|
| Time, | % release | (Ph = 2.2), 0.01M | pH = 7.4, phosphate- |
| minutes | in water | HCl | buffered saline |
| 0 | 0 | 0 | 0 |
| 5 | 0.4 | 0.6 | 11.6 |
| 10 | 2.9 | 2.2 | 22.3 |
| 20 | 5.8 | 6 | 42.1 |
| 30 | 9.9 | 8.7 | 68.9 |
| 40 | 12 | 10.2 | 78.5 |
| 60 | 21 | 16.8 | 85 |
| 80 | 26 | 19.5 | 89.3 |
| 100 | 30 | 22 | 90.7 |
| 120 | 35 | 26.7 | 95.7 |
| 140 | 40 | 29.8 | 98.6 |
| 160 | 51.3 | 34 | 100 |

### Example 4

### Dissolution test for Example 2

100 mg (equivalent to 10 mg loratadine) of the spray dried powder from Example 2 was dispersed into 900 ml dissolution media (either distilled water, HCl solution at a pH=2.2, or phosphate buffer solution at a pH=7.2 respectively) with overhead paddle stirring at 50 rpm and a temperature of 37° C. (of the dissolution media). Aliquots of each solution were taken using a pipette (1 ml Eppendorf pipette) at 5 min, 10 min, 20 min, etc. The dispersions were then diluted with ethanol for UV characterization (1 ml ethanol was added into 1 ml dispersion). The dissolution of Example 2 in different media is summarized in Table 2.

TABLE 2

| | | % release in acid | % release in buffer, |
|---|---|---|---|
| Time, | % release | (pH = 2.2), 0.01M | pH = 7.4, phosphate- |
| minutes | in water | HCl | buffered saline |
| 0 | 0 | 0 | 0 |
| 5 | 0.41 | 0.23 | 12.3 |
| 10 | 2.78 | 2.2 | 24.3 |
| 20 | 5.2 | 5.3 | 46.2 |
| 40 | 10.6 | 8.1 | 65 |
| 60 | 12.9 | 9.2 | 72.4 |
| 90 | 15 | 10.6 | 79 |
| 120 | 20.2 | 12.6 | 86.1 |
| 150 | 26.8 | 14 | 90.7 |
| 180 | 30 | 16.8 | 91.2 |
| 210 | 32.2 | 18.3 | 98.6 |
| 240 | 36 | 19 | 100 |

1. A substantially solvent-free nano-dispersion of an active in a carrier, said carrier comprising a water-soluble carrier and an enteric carrier that is soluble at intestinal pH, but insoluble at stomach pH, wherein the enteric carrier comprises at least 50% by weight of the nano-dispersion, the active comprises at least 10% by weight of the nano-dispersion and the average particle size of the active is less than 1 μm.

2. A substantially solvent-free nano-dispersion according to claim 1, wherein the active is a pharmaceutical or a nutraceutical active.

3. A dispersion according to claim 1, wherein the enteric carrier is selected from the group consisting of cellulose acetate phthalate, cellulose acetate trimellitate, hydroxypropylmethyl cellulose phthalate, hydroxypropylmethyl cellulose acetate succinate, carboxymethylethyl cellulose, polyvinyl alcohol acetate phthalate and copolymers of methacrylic acid and ethyl acrylate, and mixtures thereof.

4. A dispersion according to claim 1, wherein the carrier further comprises one or more additional carriers.

5. A process for the production of a composition comprising an active, which process comprises the steps of:
(a) providing a mixture comprising:
(i) said active,
(ii) a water-soluble carrier, and
(iii) an enteric carrier that is soluble at intestinal pH, but insoluble at stomach pH, and
(iv) a solvent for each of the active, the water-soluble carrier and the enteric carrier, and
(b) drying the mixture to remove the, or each, solvent and obtain a substantially solvent-free nano-dispersion of said active in said water-soluble and enteric carriers, wherein the enteric carrier comprises at least 50% by weight of the nano-dispersion, the active comprises at least 10% by weight of the nano-dispersion and the average particle size of the active is less than 1 μm.

6. A process according to claim 5, wherein the active is a pharmaceutical or a nutraceutical active.

7. A process according to claim 5, wherein the enteric carrier is selected from the group consisting of cellulose acetate phthalate, cellulose acetate trimellitate, hydroxypropylmethyl cellulose phthalate, hydroxypropylmethyl cellulose acetate succinate, carboxymethylethyl cellulose, polyvinyl alcohol acetate phthalate and copolymers of methacrylic acid and ethyl acrylate, and mixtures thereof.

8. A process according to claim 5, wherein the mixture further comprises optionally one or more additional carriers.

9. A process according to claim 5, wherein the active is a water-soluble active, which process comprises the steps of:
(a) providing the mixture by forming an emulsion comprising:
(i) a solution of the water-soluble active and the water-soluble carrier in water, and
(ii) a solution of the enteric carrier in a water immiscible solvent for the same, and,
(b) drying the emulsion to remove water and the water-immiscible solvent to obtain the substantially solvent-free nano-dispersion of the active in the water-soluble and enteric carriers, wherein the enteric carrier comprises at least 50% by weight of the nano-dispersion, the active comprises at least 10% by weight of the nano-dispersion and the average particle size of the active is less than 1 μm.

10. A process according to claim 9, wherein the emulsion further comprises one or more additional carriers.

11. A process according to claim 5, which process comprises the steps of:
(a) providing the mixture by forming a single phase mixture comprising:
(i) at least one non-aqueous solvent
(ii) optionally, water
(iii) said enteric carrier soluble in the mixture of (i) and (ii) and
(iv) said water-soluble carrier soluble in the mixture of (i) and (ii), and
(v) said active which is soluble in the mixture of (i) and (ii), and,
(b) drying the solution to remove any water and the water miscible solvent to obtain the substantially solvent-free nano-dispersion of the active in the water-soluble and enteric carriers, wherein the enteric carrier comprises at least 50% by weight of the nano-dispersion, the active comprises at least 10% by weight of the nano-dispersion and the average particle size of the active is less than 1 μm.

12. A process according to claim 11, wherein the single phase mixture further comprises one or more additional carriers soluble in the mixture of (i) and (ii).

13. A pharmaceutical product or composition comprising a substantially solvent-free nano-dispersion according to claim 1.

14. A nutraceutical product or composition comprising a substantially solvent-free nano-dispersion according to claim 1.

15. A delayed release composition comprising the substantially solvent-free nano-dispersion according to claim 1.

16. The of a product or composition according to claim 13 wherein the product or composition is a delayed release product or composition.

17. A kit comprising the substantially solvent-free nano-dispersion according to claim 1.

18. A process according to claim 5, wherein the drying step comprises spray-drying.

19. A process according to claim 9, wherein the drying step comprises spray-drying.

20. A process according to claim 11, wherein the drying step comprises spray-drying.
